(19) United States
(12) Patent Application Publication
(10) Pub. No.: US 2025/0101086 A1
(43) Pub. Date: Mar. 27, 2025

(54) MONOCLONAL ANTIBODY SPECIFIC FOR IR4 OR IR6 PEPTIDES

(71) Applicant: Research Foundation of the City University of New York, New York, NY (US)

(72) Inventors: Brian M. Zeglis, New York, NY (US); Weigang Qiu, Kew Gardens, NY (US)

(21) Appl. No.: 18/473,875

(22) Filed: Sep. 25, 2023

(51) Int. Cl. C07K 16/12; G01N 33/569

(52) U.S. Cl. CPC C07K 16/12; G01N 33/56911

(57) ABSTRACT

Monoclonal antibodies and antibody fragments that selectively bind to invariant region 4 or 6 (IR4, IR6) of a VlsE (variable major protein-like sequence, expressed) protein. The VlsE protein is found on the surface of multiple variants of spirochete bacteria that cause Lyme disease. The protein is abundantly and continuously expressed throughout the bacteria's time in its vertebrate host and thus detection over the entire infection is enabled. A label may be attached to enable imaging of the bacteria.

### STATEMENT OF FEDERALLY SPONSORED RESEARCH OR DEVELOPMENT

This invention was made with Government support under grant numbers AI139782 and EB030275 awarded by the National Institute of Health. The government has certain rights in the invention.

### REFERENCE TO A SEQUENCE LISTING

This application contains a Sequence Listing in computer readable form. The computer readable form is incorporated herein by reference. The computer readable file is named Sequence.xml and was created on Sep. 21, 2023 (17 KB).

### BACKGROUND OF THE INVENTION

Lyme disease is a multistage, tick-transmitted infection caused by spirochetes of the bacterial species complex Borrelia burgdorferi sensu lato (Bbsl), known more concisely (albeit controversially) as a new genus Borreliella. Lyme disease is the most common tick-borne disease in regions of North America, Europe and Asia. In the United States approximately 476,000 cases are diagnosed annually. Most Lyme disease cases in the U.S. are caused by the single species B. burgdorferi and transmitted by the hard-bodied Ixodes scapularis or I. pacificus ticks, although the same tick vectors carry other Borreliella species as well as Borrelia species closely related to relapsing fever spirochetes. B. burgdorferi causes multisystemic manifestations in humans including erythema migrans (EM) at early stages, arthritis, carditis, neuroborreliosis in late stages, and chronic symptoms associated with persistent infections.

Antigenic variation via continuously altering the sequences of surface antigens during infection is a common strategy that microbial pathogens employ to escape the adaptive immune responses of vertebrate hosts. In the two sister spirochetal groups—Borrelia causing relapsing fever and Borreliella causing Lyme disease, two homologous but distinct molecular systems have evolved facilitating continuous antigenic variation through recombination between an expressed locus and silent archival loci during persistent infection within the vertebrate hosts. In B. burgdorferi, the molecular system able to generate antigenic variation consists of one expression site (vlsE, variable major protein-like sequence, expressed) and a set of tandemly arranged silent cassettes that share more than 90% similarities to the central cassette region of vlsE. During mammalian infection, vlsE continuously expresses and undergoes random segmental recombination with the silent cassettes, generating a considerable number of new VlsE antigen variants to prolong spirochete infection in hosts.

The vlsE gene encodes a 36 kDa lipoprotein that is anchored to the outer membrane on the cell surface. The primary structure of VISE comprises the N- and C-terminal domains, as well as the central cassette, which has six highly variable regions (VR1-VR6), interspersed with six conserved, invariant regions (IR1-IR6) (see FIG. 1). The N- and C-terminal regions do not undergo antigenic variation and are thought to be important in maintaining the functional structure of the molecule. The cassette sequences undergo antigenic variation during infection. The crystal structure of recombinant VlsE protein revealed that the six VRs constitute loop structures and form a dome on the membrane distal surface exposed to the host environment, which may shield the IRs from antibody binding.

VlsE elicits strong humoral responses that can be detected throughout Lyme disease, making it a powerful antigen in serologic assays of Lyme disease diagnosis. Contrary to the established paradigm of weak immunogenicity of the conserved regions of bacterial surface proteins, the conserved IR6 elicits immunodominant antibody responses during human infection despite the region being largely inaccessible on the intact VISE molecule. The surprising finding of immunodominance of IR6 in human patients is hypothesized to be a result of antigen processing of the VlsE proteins in nonreservoir host species.

A 26-amino acid peptide that reproduces the IR6 sequence, known as the C6 peptide, is used in commercial diagnostic tests for Lyme disease. The standardized two-tiered testing (STTT) for Lyme disease diagnosis includes a screening enzyme immunoassay (EIA) with the whole-cell sonicate and a subsequent confirmatory Western blot assay for the presence of both IgM and IgG antibodies against ten Borreliella antigens. Recently, a modified two-tiered testing (MTTT) protocol using two sequential EIAs with C6 peptide or the whole VlsE protein has been developed. MTTT improved sensitivity and specificity relative to STTT, especially in Lyme patients with early-stage manifestations. Nevertheless, the overall sensitivity for early-stage diagnosis remains low, ranging from 36% to 54%, even with MTTT.

During the transmission cycle of B. burgdorferi, the vis locus is expressed during the late-stage persistent infection within the mammalian host, in contrast to genes like ospA (encoding outer surface protein A) expressed within the ticks, and genes like ospC expressed exclusively during a short window of time when the spirochetes begin to migrate from the tick to the mammalian host. As a multicopy gene family and driven by diversifying natural selection, the silent vis cassettes exhibit high sequence variability not only between B. burgdorferi strains but also within the same genome.

Currently, both diagnostic assays are indirect tests and do not distinguish between active infection and past exposure. Accordingly, there is a need to simplify the testing protocol for Lyme disease, improve testing sensitivity in the early infection stage, and detect the presence of the Lyme pathogen or its derivative antigens directly.

The discussion above is merely provided for general background information and is not intended to be used as an aid in determining the scope of the claimed subject matter.

### SUMMARY

This disclosure provides monoclonal antibodies and antibody fragments that selectively bind to invariant region 4 or 6 (IR4, IR6) of a VlsE (variable major protein-like sequence, expressed) protein. The VlsE protein is found on the surface of multiple variants of spirochete bacteria that cause Lyme disease. The protein is abundantly and continuously expressed throughout the bacteria's time in its vertebrate host and thus detection over the entire infection is enabled. A label may be attached to enable imaging of the bacteria.

The technical problem to be solved is the enablement of detection and imaging of Bbsl bacteria despite the heterogeneity of Bbsl strains. A further technical problem to be solved is the enablement of the direct detect of the Bbsl bacteria without relying on indirect detection techniques. A still further technical problem to be solved is the detection of Bbsl bacteria throughout the lifecycle of the infection.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 shows the gene structure of the vis locus in B. burgdorferi strain B31. The vis locus is located close to the telomere of the linear plasmid lp28-1 (GenBank accession AE000794) in the B31 genome, with cassettes of silent (unexpressed) open reading frames (ORFs) (vls2 through vls16) and an expressed ORF (vlsE) containing the vls1 cassette introduced by recombination. Dashed arrows indicate the direction of coding strands.

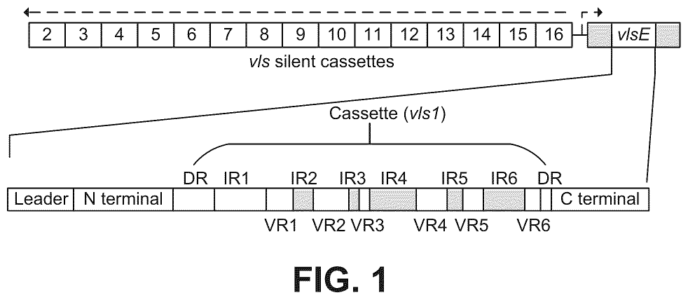

FIG. 2 shows R-highlighted three-dimensional structures of VISE. Structure diagrams of VlsE protein from B31 were prepared in Chimera (Version 1.15) based on the PDB file (accession 1L8W). IRs were highlighted in different colors (IR1 in green, IR2 in yellow, IR3 in red, IR4 in dark blue, IR5 in magenta, and IR6 in cyan). (Panel A) Ribbon diagram showing that IRs tend to form alpha helices. (Panel B) Surface-filled diagram showing membrane surface exposure of IRs in monomeric form. (Panel C) and (Panel D) Dimerized structure models. The structures were oriented to show the membrane-proximal part at the bottom (Panels A, B, and C).

FIG. 3 is a schematic depiction of a monoclonal antibody showing a pair of heavy and light chains. The heavy chains have a variable heavy chain (V_{H}) and the light chains have a variable light chain (V_{L}).

FIG. 4 is a table depicting the primary structure of the variable heavy chains (V_{H}) and the variable light chains (V_{L}) for four different monoclonal antibodies.

FIGS. 5A-SF are graphs showing the results of antigenicity testing of conserved epitopes against host sera.

FIG. 6 shows rabbit B cells produce IR-specific antibodies. (Bar plot) Twenty VlsE-positive cell lines (x-axis) from rabbits immunized with recombinant VlsE were selected with the B cell sorting technique and tested against purified antigens with ELISA. Bovine serum albumin (BSA) was used as the negative control and the purified recombinant VISE protein (of strain B31) as the positive control. An OD₄₅₀ value (y-axis) greater than 3 standard deviations above the mean BSA reactivity (dashed lines) was considered to show significant antibody-antigen reactivity. Five cell lines expressing anti-IR4 antibodies and one cell line expressing anti-IR6 antibodies were identified. (Inset) SDS-PAGE image of induction and purification of the recombinant B31 VlsE. Elution 1 and Elution 2 were combined into a single preparation with an estimated purity of about 65% and a concentration of about 5.0 mg per mL, which was subsequently used to immunize New Zealand rabbits. “Pre-ind,” pre-IPTG induction; “Post-ind,” postinduction; “Flo-through,” flowthrough sample; “Wash,” wash sample; “Elu1” and “Elu2,” elution samples.

FIG. 7 is a graph showing the results of titration experiments with the monoclonal antibodies. Serially diluted preparations of the four affinity-purified IR-specific rMAbs cloned from rabbits immunized with recombinant VlsE were tested with ELISA against their respective IRs (1D11 against IR6; 15E2, 17A8, and 28D3 against IR4). The R package drc (Version 3.0-1) was used to estimate the effective concentration and to plot the titration curves. EC50 (effective concentration at 50% of the maximum activity) values were estimated from the fitted curves, with a lower EC50 indicating stronger antigen affinity.

FIG. 8A provides schematic depictions of labeled antibodies.

FIG. 8B provides schematic depictions of labeled single-domain antibodies.

FIG. 8C provides schematic depiction of a labeled fragment antibody.

FIG. 9 is a graph showing results of immunoreactivity experiments with beads bearing recombinant VlsE. These results revealed that both radioimmunoconjugates exhibited immunoreactive fractions of about 90%.

### DETAILED DESCRIPTION OF THE INVENTION

VlsE (variable major protein-like sequence, expressed) is an outer surface protein of the Lyme disease pathogen (Borreliella species) responsible for its within-host anti-genic variation and a key diagnostic biomarker of Lyme disease. However, the high sequence variability of VlsE poses a challenge to the development of consistent VlsE-based diagnostics and therapeutics. The standard diagnostic protocols detect immuno-globins elicited by the Lyme pathogen, not the presence of the pathogen or its derived antigens. This disclosure provides recombinant monoclonal antibodies (rMAbs) and single-domain antibodies (sdAb) that bound specifically to conserved epitopes on VlsE. Advantageously, the disclosed antibodies permit detection of the Borreliella bacteria throughout the infection lifecycle.

Amino acid sequence variability encoded by the vis genes from thirteen B. burgdorferi genomes were examined by evolutionary analyses. Broad inconsistencies of the sequence phylogeny with the genome phylogeny were found, indicating rapid gene duplications, losses, and recombination at the vis locus.

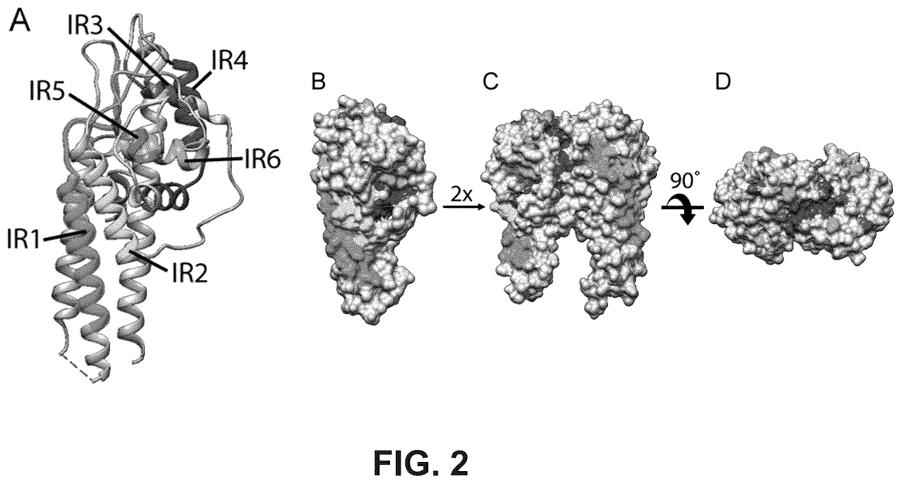

Referring to FIG. 2, IR1 to IR6 were mapped to a published three-dimensional structure of the VISE protein (from the strain B31, see J Biol. Chem. Vol. 277, 24, p 21691-21696, June 202 to Christoph Eicken et al.). All the IRs formed alpha helixes, as the ribbon model showed (panel A). The space-filled model showed that IR1, IR2, and IR4 were partially surface exposed while IR3, IR5, and IR6 exhibited limited surface exposure (panel B)). The VISE molecules likely form dimers on the spirochete cell surface which would further shield the invariant regions located on the monomer-monomer interface (panels C and D). Nevertheless, IR4 is partially exposed at the membrane distal surface even in a dimerized form (panel D).

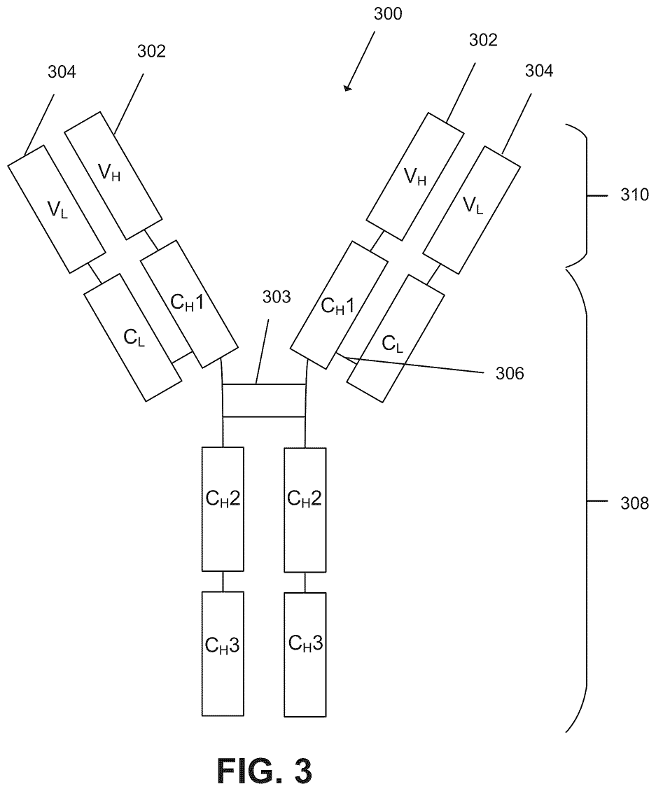

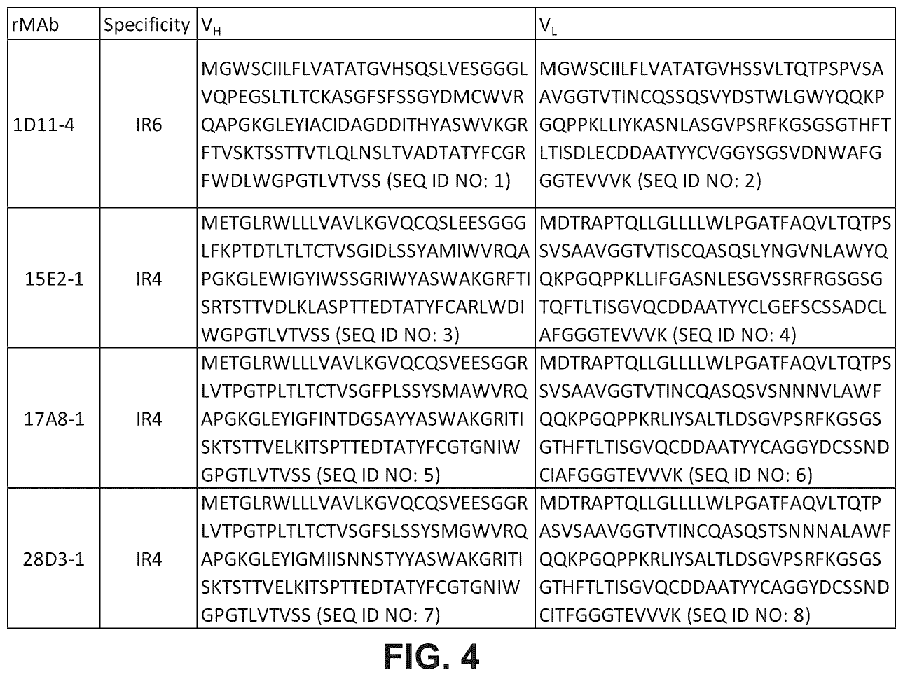

Referring to FIG. 3, an antibody 300 is depicted that comprises a means for selectively binding to IR4 or IR6. The antibody 300 comprises a pair of heavy chains 302 that are joined by disulfide bonds 303. The antibody 300 also comprises a pair of light chains 304 that are joined to respective heavy chains by disulfide bonds 306. Each heavy chain 302 and each light chain 304 has a constant region 308 (at a C-terminus of the chain) and a variable region 310 (at an N-terminus of the chain). The variable region 310 includes a variable heavy chain (V_{H}) and variable light chain (V_{L}). The V_{H} and V_{L} of the four rMAbs are identified in FIG. 4 and are specifically designed to provide a means for selectively binding to IR4 or IR6. Specifically, when VA is SEQ ID NO: 1 and V_{L} is SEQ ID NO: 2, the variable region 310 provides a means for selectively binding to IR6. When V_{H} is SEQ ID NO: 3 and V_{L} is SEQ ID NO: 4, the variable region 310 provides a means for selectively binding to IR4. When V_{H} is SEQ ID NO: 5 and V_{L} is SEQ ID NO: 6, the variable region 310 provides another means for selectively binding to IR4. When VA is SEQ ID NO: 7 and V_{L} is SEQ ID NO: 8, the variable region 310 provides yet another means for selectively binding to IR4. Data in support of these findings are provided elsewhere in this disclosure.

The antibody 300 has a molecular weight between 100 kDa and 200 kDa. In another embodiment, the antibody 300 has a molecular weight between 120 kDa and 180 kDa. In yet another embodiment, the antibody 300 has a molecular weight between 140 kDa and 160 kDa. The constant region 308 comprises the constant light region (Ct) and the constant heavy regions (C_{H}1, C_{H}2 and C_{H}3). Details concerning the constant region 308 from rabbits may be found in Exp. Mol Med. 2017 March; 49(3) e305 to Weber et al.

### Preliminary Antigenicity Testing Using Host Sera

To identify conserved epitopes, five long conserved invariant regions (IRs) on VlsE were synthesized as shown in Table 1. The antigenicity of these five IR peptides were tested using sera from three mammalian host species including human patients, the natural reservoir white-footed mouse (Peromyscus leucopus), and VISE-immunized New Zealand rabbits (Oryctolagus cuniculus) using an enzyme-linked immunosorbent assay (ELISA).

TABLE 1
Peptides used to screen for IR-specific monoclonal antibodies

| Peptide (a) | Sequence (b) |
|---|---|
| IR1 | EVSELLDKLVKAVKTAEGASSG (SEQ ID NO: 9) |
| IR2 | (ASVK)GIAKGIKEIVEAA(GGSE) (SEQ ID NO: 10) |
| IR3 (c) | AGKLFVK (SEQ ID NO: 11) |
| IR4 | KAAGAVSAVSGEQILSAIV(TAA) (SEQ ID NO: 12) |
| IR5 | (AEEAD)NPIAAAIG(TTNEDA) (SEQ ID NO: 13) |
| IR6 | MKKDDQIAAAIALRGMAKDGKFAVK (SEQ ID NO: 14) |

(a) IRs: Invariant regions. Six IRs were identified from an alignment of VlsE proteins from three strains representing two species
(b) IR sequences were based on the VlsE protein of the B31 strain (GenBank accession U76405). Flanking residues (in parentheses) were padded to the IR2 and IR4 to facilitate ELISA. For IRS, the padded residues were the most common residues flanking this conserved region
(c) Antigenicity of IR3, the shortest IR, was not tested

TABLE 2
sequences of the target invariant regions (IR1-IR6)

| Region | Sequence |
|---|---|
| IR1 | EVSELLDKLVKAVKTAEGASSG (SEQ ID NO: 9) |
| IR2 | GIAKGIKEIVEAA (SEQ ID NO: 15) |
| IR3 (c) | AGKLFVK (SEQ ID NO: 11) |
| IR4 | KAAGAVSAVSGEQILSAIV (SEQ ID NO: 16) |
| IR5 | NPIAAAIG (SEQ ID NO: 17) |
| IR6 | MKKDDQIAAAIALRGMAKDGKFAVK (SEQ ID NO: 14) |

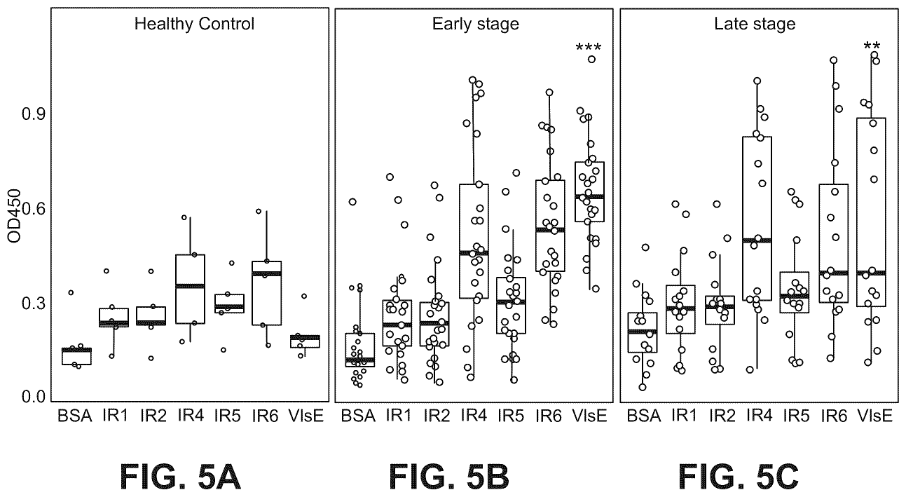

Referring to FIGS. 5A-F, the antigenicity of five IRs (x-axis) was quantified with ELISA (see Materials and Methods). Bovine serum albumin (BSA) was used as the negative control and the purified recombinant VlsE protein (of strain B31) as the positive control. Each IR peptide was tested for reactivity (optical density at 450 nm (OD₄₅₀), y-axis) with host sera (represented by dots). Reactivity with forty six human sera, including those from four healthy controls (FIG. 5A), twenty five early-stage Lyme disease patients (FIG. 5B), and seventeen late-stage Lyme disease patients (FIG. 5C). The antigens reacted significantly stronger with the patient sera than with the control sera for both the early-stage and late-stage samples. Reactivity of individual IRs, however, was not significant between the patient and control. VISE reacted strongly with both the early and late-stage patient samples relative to the control sera. Reactivity with forty six patients (FIG. 5D), ten naturally infected reservoir hosts (white-footed mouse, Peromyscus leucopus) (FIG. 5E), and four New Zealand rabbits (Oryctolagus cuniculus) immunized with recombinant VlsE (FIG. 5F). Asterisks indicate levels of statistical significance: *, 0.01<P<0.05; **, 0.001<P<0.01; ***, P<0.001.

The IR4 and IR6 peptides were the most antigenic and reacted strongly with both the human and rabbit sera, while all IR peptides reacted poorly with sera from natural hosts. For the forty six human sera, reactivities of the IR4 and IR6 peptides with the human sera were significantly higher than that of bovine serum albumin (BSA) (P=2.87e−13 and 3.06e−13 by analysis of variance [ANOVA], respectively), while reactivities of IR1, IR2, and IR5 were less significant (P=0.034, 0.034, and 0.0019 by ANOVA, respectively) (FIG. 5A and FIG. 5D). Reactivity of VISE recombinant protein was the strongest (P, 2.2e−16 by ANOVH). In addition, reactivities of IR4 and IR6 with the human sera were weakly, although significantly, correlated with those of the VISE (P=7.6e−4 and R²=0.212 for IR4, P=3.6e−3 and R²=0.158 for IR6, both by linear regression). Reactivities of both early and late-stage samples were significantly higher than the non-Lyme control samples (P=8.49e−5 and P=2.63e−4, respectively, by t test) but there was no significant difference in reactivity between the early and late-stage patient samples (P=0.8654 by t test).

For sera from ten naturally infected white-footed mice, the natural reservoir host of B. burgdorferi, the IR peptides showed weakly significant differences in antigenicity among the antigens (P=0.0159 by ANOVH), with only VISE showing a significant difference from the BSA control (P=2.9e−3 by ANOVH) (FIG. 5E).

The gross anti-VISE polyclonal antibodies were extracted from the serum of four immunized rabbits. Reactivities of the IRs against the rabbit polyclonal antibodies showed a similar pattern as those against the naturally infected human (FIG. 5F). For example, VlsE, IR4, and IR6 displayed the highest antigenicity (P=6.8e−10, 1.2e−7, and 2.1e−8, respectively, with an overall P=2.2e−10 by ANOVH). Antigenicity of the IR1, IR2, and IR5 peptides against the rabbit polyclonal antibodies did not differ or differed weakly from that of BSA, the negative control (P=0.855, 0.011, and 0.236 by ANOVA, respectively). The preimmunized rabbit sera did not react with any antigens (P=1.76e−6 by paired t test between the preimmunized and post-immunized sera).

These ELISA results suggested that (i) anti-VlsE antibodies were present in patients throughout different stages of Lyme disease, (ii) antibodies against the VISE IRs were strongly present in naturally infected or artificially immunized non-reservoir hosts but minimally present in reservoir hosts, and (iii) the IR4 and IR6 peptides were highly immunogenic conserved epitopes on the VISE molecule in non-reservoir hosts relative to the IR1, IR2, and IR5 peptides. These results were consistent with the conclusions of earlier studies on the antigenicity of VISE and conserved epitopes, which established the use of VlsE and the C6 peptide (derived from IR6) in both the standard and modified diagnostics tests for Lyme disease.

These data establish IR4 peptide was as antigenic as the IR6 peptide. Indeed, both IR peptides reacted at a level similar to the reactivity of the whole VlsE protein with the sera from naturally infected and immunized hosts. The use of the highly conserved IR4 and IR6 as targets for theragnostic agents has the advantage that they are expected to exhibit antigenicity against a broad set of B. burgdorferi strains, with the potential to mitigate the challenge of strain-specific antigenicity of the highly variable antigens including VISE and OspC.

rMAbs Specific to IR4 and IR6

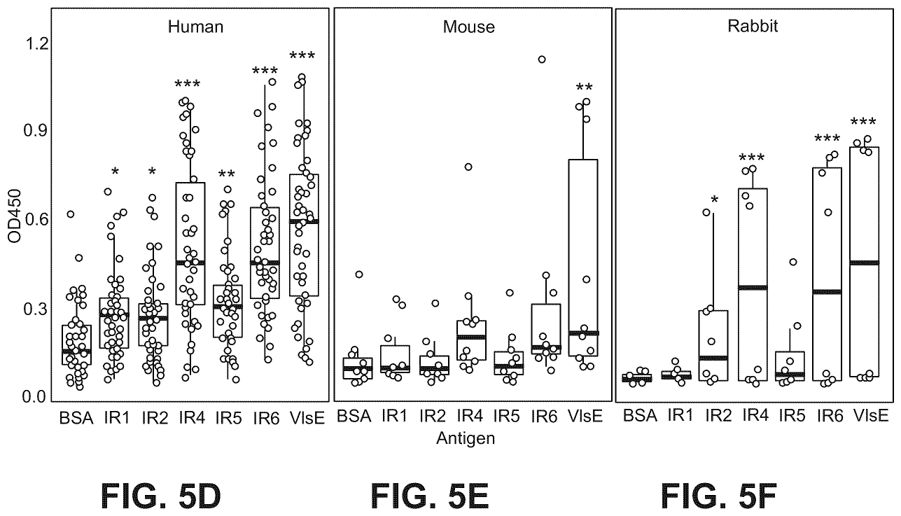

Recombinant VISE of the strain B31 was overexpressed, purified and used to immunize New Zealand rabbits (FIG. 6, gel image). IR-specific antibodies were identified via B cell sorting and by testing the reactivity of the supernatant of twenty B cell lines against the five IR peptides (see Table 1) with ELISA. One cell line (1D11) bound specifically to the IR6 peptide and five cell lines (7C9, 15E2, 17A8, 28D3, and 42G10) specifically to the IR4 peptide in addition to their binding to the recombinant VlsE protein (FIG. 6, bar plots). Supernatants of the remaining fourteen B cell lines reacted with VlsE but not with the IR peptides, suggesting that most B cell lines in the immunized rabbit expressed antibodies recognizing epitopes located on the variable but not the conserved regions.

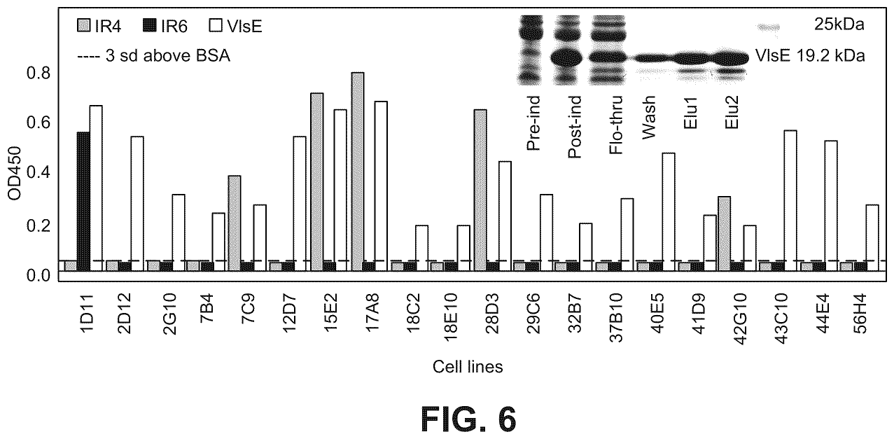

One pair of the most abundant heavy chain and light chain variable region (V_{H} and V_{L}) sequences in each of four IR-specific cell lines-including the anti-IR6 ID11 cell line and three top anti-IR4 cell lines-were identified by pyrosequencing and subsequently cloned and overexpressed. Specificities of the purified recombinant monoclonal antibodies (rMAb) were validated using ELISA. The initial rMAb cloned from the 1D11 cell line based on the most abundant V_{H} and V_{L} sequences was not reactive to the IR6 peptide as the supernatant of the cell line did. A new rMAb-based on the second most abundant V_{H} and V_{L} sequences—was re-cloned and overexpressed and reacted with the IR6 peptide strongly and specifically. The V_{H} and V_{L} sequences of the four IR-specific rMAbs are shown in FIG. 4 and their binding characteristics were obtained by titration experiments (FIG. 7).

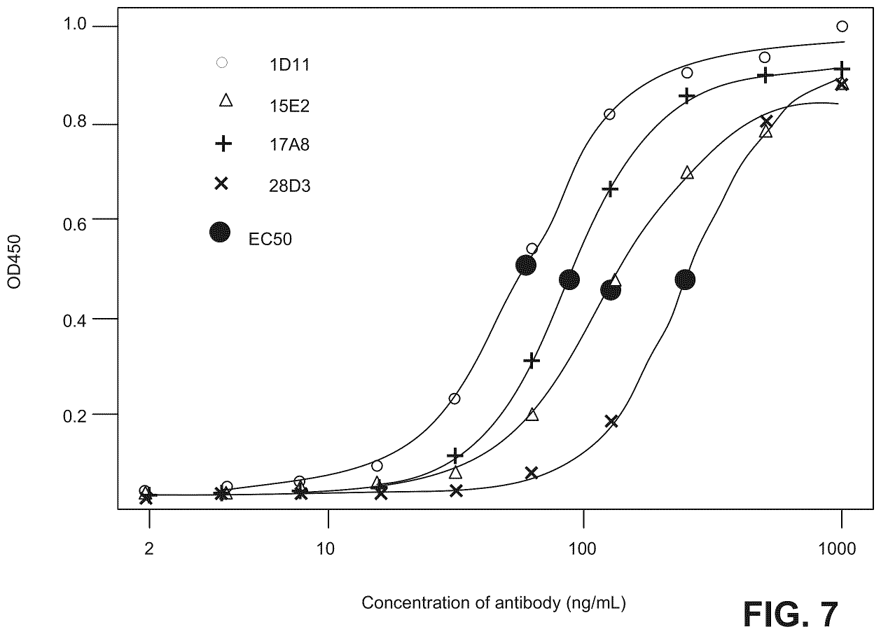

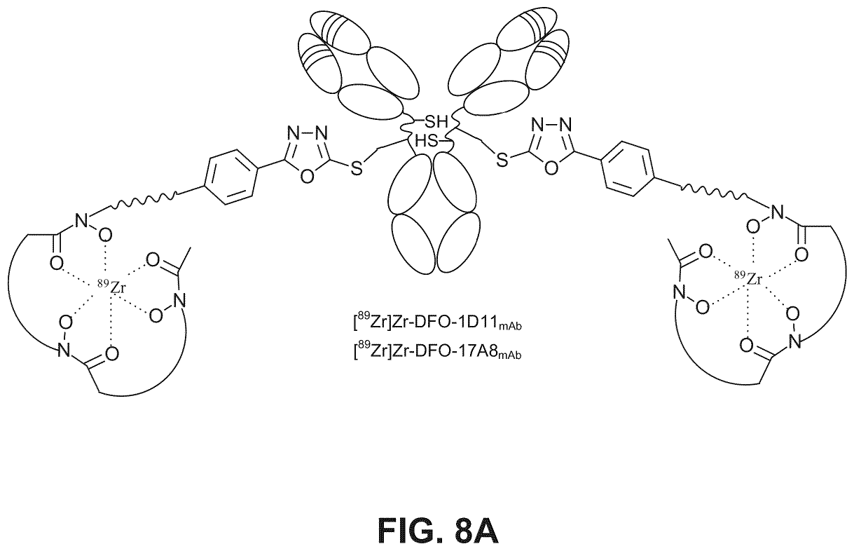

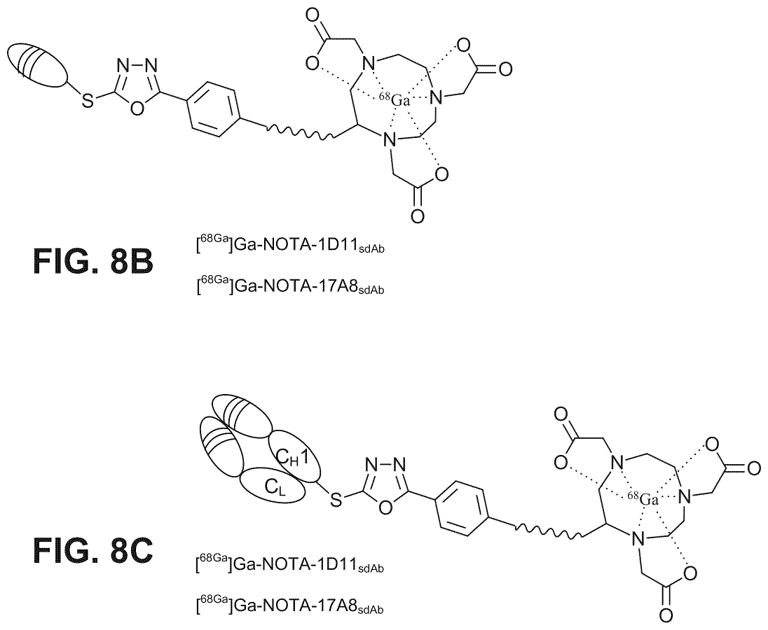

In another embodiment, the disclosed monoclonal antibodies are used in conjunction with a covalently bound label, such as a radiolabel or a fluorescent label. Such labels are useful in medical imaging (e.g. positron emission tomography (PET), etc.). Examples of suitable labels include chelators such as desferrioxamine (DFO), 1,4,8,11-tetraazacyclotetradecane-N,N′,N″,N′″-tetraacetic acid (TETA), 1,4,7,10-Tetraazacyclododecane-1,4,7, 10-tetraacetic acid (DOTA), tetrazine, 1,4,7-triazacyclononane-N,N′,N″-triacetic acid (NOTA), 1,4,7-triazacyclononane, 1-glutaric acid-4,7-acetic acid (NODAGA), 1,4,7-triazacyclononane-1,4-diacetate (NODA), diethylenetriaminepentaacetic acid (DTPA), 1,4,8,11-tetraazacyclotetradecane-N,N′,N″,N′″-tetraacetic acid (TETA), 1,4,8, 11-Tetraazabicyclo [6.6.2]hexadecane-4,11-diacetic acid (CB-TE2A), hydroxybenzyl ethylenediamine (HBEd), and 1,8-Diamino-3,6, 10, 13, 16, 19-hexaazabicyclo [6,6,6]-eicosane (Diam Sar), (with metals such as Zr-89, Cu-64, Ga-68, In-111, I-124, I-131, Lu-177) and fluorophores. For example, and with reference to FIG. 8A, the antibody from 1D11 or 17A8 cells lines may be labeled with a Zr-89 ion chelated by DFO. Alternatively, the antibody from 1D11 or 17A8 cells lines may be labeled with a Ga-68 ion chelated by NOTA. Alternatively, as shown in FIG. 8B, the label may be attached to the variable region only (e.g. V_{H} or V_{L}) to provide a single-domain antibody (sdAb). In one embodiment, the antibody consists of the single-domain antibody and a label. In the embodiment of FIG. 8C, the label is attached to a fragment antibody (Fab). In one embodiment, the antibody consists of the two variable regions (V_{H} and a V_{L}) plus a label. In FIG. 8C, the Fab comprises both a V_{H} and a V_{L} selected from SEQ ID NO: 1 to 8, as well as the constant light (C_{L}) region and the first constant heavy (C_{H}1) region. In one embodiment, the antibody consists of the two variable regions (V_{H} and a V_{L}), the two constant regions (C_{H}1 and C_{L}). Generally, the antibody fragments has a molecular weight of less than 100 kDa, inclusive of the weight of the label. In another embodiment, the antibody fragment has a molecular weight of less than 50 kDa.

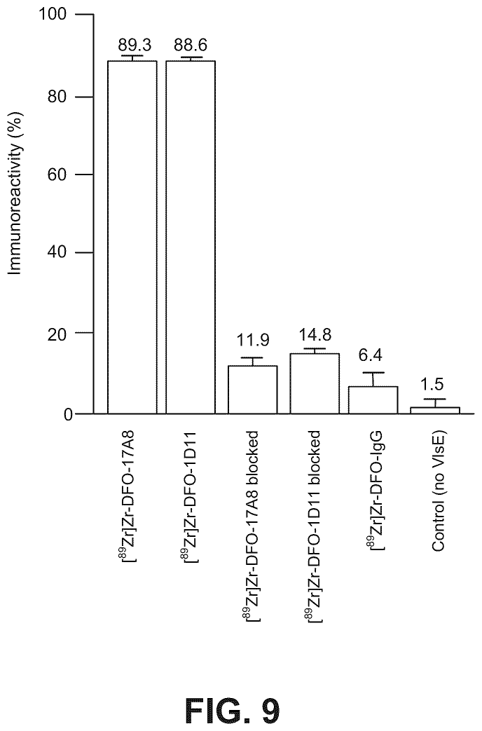

The resulting immunoconjugates were shown to have degrees of labeling of 2.0 DFO per mAb and 1.9 DFO per mAb, respectively, and exhibited greater than 95% stability to aggregation over 7 d in serum at 37° C. Importantly, the binding of each mAb to VlsE was not substantially perturbed by the modification. Labeled DFO-17A8 displayed a ED 50 of 22.1 ng/mL for \′IsE (compared to 18.8 ng/ml for 17A8), while labeled DFO-1D11 displayed a ED50 of 24.8 ng/ml for VlsE (compared to 10.9 ng/mL for 1D11). Both DFO-1D11 and DFO-17A8 were radiolabeled with [89Zr]Zr4+ to produce [89Zr]Zr-DFO-1D11 and [89Zr]Zr-DFO-17A8 in high yield, purity, and specific activity (greater than 10 mCi/mg for each). Furthermore, radio-thin layer chromatography (radioTLC) and radio-size exclusion chromatography (radioSEC) confirmed that both radioimmunoconjugates were stable to demetallation and aggregation over 7 d in serum at 37° C. Immunoreactivity experiments with beads bearing recombinant V′IsE revealed that both radioimmunoconjugates exhibited immunoreactive fractions of about 90% (FIG. 9).

The monoclonal antibodies from the 1D11 and 17A8 cell lines were also site-specifically modified using a bifunctional variant of DFO bearing a thio-reactive phenyloxadiazolyl methyl sulfone moiety (PODS). See U.S. Patent Publication US2022/0024904 to Zeglis, the content of which is incorporated by reference.

A biological sample may be exposed to a solution of the labeled monoclonal antibody (or sdAb). The label selectively binds to the IR4 or IR6 regions of any bacteria in the biological sample and thus permits imaging of the bacteria. Examples of biological samples include, blood samples, salvia samples, tissue samples, etc.

### Materials and Methods

Identification of vls cassette sequences and evolutionary analysis: The whole-genome sequences of thirteen B. burgdorferi strains was downloaded from NCBI GenBank. The silent cassette sequences of the B31-5A3 clone (GenBank accession U76406) were used as the queries to search for sequences homologous to the vis cassette sequences using HMMER (version 3.3.2). A customized web-based software tool was developed to identify and extract individual vis sequences given a B. burgdorferi replicon sequence. Sequences of the silent cassettes and vlsE were translated, aligned, and converted into a codon alignment using MUSCLE (version 3.8.31) and the bioaln utility (−dna2pep method) of the BpWrapper (version 1.13) toolkit. A maximum likelihood tree was subsequently inferred using IQ-TREE (version 1.6.1) with the best-fit nucleotide substitute model KOSI07 and 1000 bootstrap replicates. Branches with lower than 80% bootstrap support were collapsed using the biotree utility (−D method) of the BpWrapper toolkit. The tree was rendered using the R package ggtree (Version 2.2.4). To quantify the sequence conservation, evolutionary rates at individual amino acid positions were estimated using Rate4Site (version 3.0.0) with the protein alignment and the phylogenetic tree as inputs and the B31-5A3 VISE sequence (GenBank accession U76405) as the reference. Sequence conservation at the IRs was further quantified and visualized with WebLogo.

Synthesis of peptides representing conserved epitopes of VISE: The preparation of the peptides was based on the annotation of the B31-5A3 VISE protein sequence. Five invariant regions, IR1, IR2, IR4, IR5, and IR6, were tested for antigenicity using sera from three host species. IR3, the shortest IR, was excluded from the antigenicity test. Extra flanking amino acids were added to IR2, IR4, and IR5 to meet the minimum length for peptide synthesis. Peptides were commercially synthesized and biotin-labeled on the N terminus using Fmoc chemistry (GenScript, Piscataway, NJ, USA). Sequences of these peptides are shown in Table 1.

Sera collection from naturally infected hosts: The 56 serum samples, consisting of Lyme patient and control sera provided by the US Centers for Disease Control and Prevention (CDC; n=40), Lyme patient sera provided by Maria Gomes-Solecki (University of Tennessee Health Science Center; n=6), and sera from naturally infected individuals of the reservoir host white-footed mouse (Peromyscus leuco-pus) originated from Millbrook, New York (n=10), have been used and described in previous publica-tions (53, 75, 76). Briefly, among the human samples, 25 serum samples were derived from patients with early-stage Lyme disease including those diagnosed as having the skin symptom erythema migran (EM) or as EM convalescence. Seventeen human serum samples were from patients displaying late-stage Lyme disease symptoms including arthritic, cardiac, and neurological Lyme diseases. Four human serum samples were from healthy individuals as controls.

Cloning, overexpression, and purification of recombinant VlsE protein. Recombinant VISE protein from the B31 strain was cloned, overexpressed, and purified using a known protocol described the academic literature (Di, L., Akther, S., Bezrucenkovas, E. et al. Maximum antigen diversification in a lyme bacterial population and evolutionary strategies to overcome pathogen diversity. ISME J 16, 447-464 (2022)) Briefly, the 585-bp vlsE cassette region (including the IR1 through VR6 regions) of the B31-5A3 clone was codon-optimized, synthesized, and cloned into the pET24 plasmid vector which then transfected Escherichia coli BL21 cells. A10× Histidine-tag was added on the N terminus of the construct to facilitate the downstream purification. All cloning work was performed by a commercial service (GeneImmune Biotechnology Corp., Rockville, MD, USA). The E. coli strain that contained a cloned vlsE cassette was cultured in Luria-Bertani (LB) broth containing 0.4% glucose and 50 mg/mL Ampicillin. When the culture reached exponential growth, expression of the cloned vlsE cassette was induced by adding isopropyl b-d-1-thiogalactopyranoside (IPTG) to a final concentration of 0.25 mM and by incubation overnight at 25° C. Cells were collected and then lysed by lysozyme and sonication. The lysate supernatant, containing the recombinant VISE protein, was purified using nickel Sepharose beads (Ni-NTA, Thermo Fisher Scientific, Waltham, MA, USA) following the manufacturer's protocol. The identity and concentration of the purified protein were examined and quantified using the sodium dodecyl sulfate-polyacrylamide gel electrophoresis (SDS-PAGE) and the Pierce Bradford Protein assay kit (Thermo Fisher Scientific, Waltham, MA, USA).

Immunization of rabbits and preparation of polyclonal and monoclonal antibodies: Antibody preparation was conducted with a commercial service GenScript (Piscataway, NJ, USA). Briefly, the project consisted of four stages. In stage 1, animals were immunized, and polyclonal antibodies were obtained. Specifically, four New Zealand rabbits (Oryctolagus cuniculus) were immunized with 100 mg purified recombinant VlsE protein on days 1, 14, and 28. The rabbits were bled for antiserum collection 1 week after the third immunization. The antisera were subsequently purified by affinity chromatography to obtain polyclonal antibodies (pAbs), which were assayed for anti-VISE activity. In stage 2, monoclonal antibodies (MAbs) were identified via single B cell sorting. Peripheral blood mononuclear cells (PBMC) were collected from the two selected immunized rabbits 1 week after a booster dose with the recombinant VlsE. Plasma B cells (CD1381) were isolated and enriched using a commercial kit. B cells were transformed by a proprietary process and then cloned by limiting dilution. The supernatants of positive cell lines were used to test for binding with VlsE and positive cell lines were chosen to produce monoclonal antibodies. In Stage 3, the variable domains of the light and heavy chains of the VISE-binding antibodies were sequenced Total RNA was isolated from the VISE-binding B cell lines and reverse-transcribed into cDNA using universal primers. DNA sequences encoding variable domains of the heavy chain and the light chain were amplified and sequenced. In Stage 4, the amplified antibody variable fragments were cloned into plasmid vector pcDNA3.4, which was then transfected into mouse cells for expression. Supernatants of cell cultures were harvested continuously. The recombinant monoclonal antibodies (rMAbs) were purified using protein A/G affinity chromatography (with immobilized protein A and G from Staphylococcus aureus) followed by size exclusion chromatography (SEC).

Identification of IR-specific MAbs with ELISA: Sera from naturally infected humans and P. leucopus were tested to evaluate reactivity to the IR peptides (Table 1) and the recombinant VlsE protein with ELISA using a protocol described previously (Di, L., Akther, S., Bezrucenkovas, E. et al. Maximum antigen diversification in a lyme bacterial population and evolutionary strategies to overcome pathogen diversity. ISME J 16, 447-464 (2022)). Briefly, a 96-well MICROLON 600 plate (USA Scientific, Inc., Ocala, FL, USA) was incubated with 10 mg per mL of antigen overnight at 4° C. Serum samples diluted between 1:100 to 1:1000 were applied after blocking with 5% milk and were incubated for 2 h at 37° C., followed by the application of horseradish peroxidase (HRP)-conjugated secondary antibodies. Goat Anti-Human IgG/IgM (H1L) (Abcam, Cambridge, UK) 1:40,000 was used for assays of human sera and the Goat anti-P. leucopus IgG (HIL) (SeraCare Life Sciences, MA, USA) 1:1000 for assays of P. leucopus sera. The antigen-antibody reaction was probed by TMB ELISA Substrate Solution (Invitrogen eBioscience) and was terminated with 1 M sulfuric acid after 15 min Binding intensities were measured at the 450 nm wavelength using a SpectraMax i3 microplate reader (Molecular Devices, LLC, CA, USA).

The same ELISA protocol was followed to test against binding with the rabbit-originated antibodies as well, including the purified anti-VlsE pAbs, the supernatants of selected B cell cultures, and the purified rMAbs. Mouse Anti-Rabbit IgG Fr secondary antibody (GenScript, Piscataway, NJ) 1:30,000 was used for assays of these rabbit-derived samples. Serial dilutions of MAbs by factors from 1,000 to 512,000 were tested with ELISA to quantify the binding activities.

Protein structure visualization” The PDB file of the VlsE protein structure (accession no. ILSW) was downloaded from the protein data bank (PDB). The PDF file describes a tetramer of VlsE. Chimera (version 1.15) was used to visualize the protein structure in ribbon and surface-filled formats and to color the six invariable regions (IR1-6).

Animal care: Antibody production from the New Zealand rabbits followed the protocols approved by the Office of Laboratory Animal Welfare (OLAW) Assurance and the Institutional Animal Care and Use Committee (IACUC) of the vendor (GenScript, Piscataway, NJ).

Data availability: Data visualization and statistical analysis were performed in the R statistical computing environment accessed with RStudio. The alignment of translated vis sequences, ELISA readings, and R scripts are publicly available on GitHub.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A composition of matter comprising
a peptide that provides a means for selectively binding to an invariant region of a VlsE (variable major protein-like sequence, expressed) protein, the invariant region being selected from a group consisting of invariant region 4 (IR4) given by SEQ ID NO: 16 and invariant region 6 (IR6) given by SEQ ID NO: 14.

2. The composition of matter as recited in claim 1, wherein the peptide is part of a monoclonal antibody.

3. The composition of matter as recited in claim 1, further comprising a label covalently bound to the peptide, the label selected from a group consisting of a radiolabel and a fluorescent label.

4. A method for labeling a bacterium, the method comprising:
exposing a biological sample to the composition of matter as recited in claim 3, wherein the biological sample comprises the bacterium.

5. The composition of matter as recited in claim 1, wherein the invariant region is invariant region 6 (IR6) and the means for selectively binding comprises a pair of heavy chains and a pair of light chains, each heavy chain is SEQ ID NO: 1 and each light chain is SEQ ID NO: 2.

6. The composition of matter as recited in claim 5, further comprising a label covalently bound to the monoclonal antibody, the label selected from a group consisting of a radiolabel and a fluorescent label.

7. A method for labeling a bacterium, the method comprising:
exposing a biological sample to the peptide as recited in claim 6, wherein the biological sample comprises the bacterium.

8. The composition of matter as recited in claim 1, wherein the invariant region is invariant region 4 (IR4) and the means for selectively binding comprises a pair of heavy chains and a pair of light chains, each heavy chain is SEQ ID NO: 5 and each light chain is SEQ ID NO: 6.

9. The composition of matter as recited in claim 8, further comprising a label covalently bound to the peptide, the label selected from a group consisting of a radiolabel and a fluorescent label.

10. A method for labeling a bacterium, the method comprising:
exposing a biological sample to the composition of matter as recited in claim 9, wherein the biological sample comprises the bacterium.

11. The composition of matter as recited in claim 1, wherein the invariant region is invariant region 4 (IR4) and the means for selectively binding comprises a pair of heavy chains and a pair of light chains, each heavy chain is SEQ ID NO: 3 and each light chain is SEQ ID NO: 4.

12. The composition of matter as recited in claim 11, further comprising a label covalently bound to the peptide, the label selected from a group consisting of a radiolabel and a fluorescent label.

13. A method for labeling a bacterium, the method comprising:
exposing a biological sample to the composition of matter as recited in claim 12, wherein the biological sample comprises the bacterium.

14. The composition of matter as recited in claim 1, wherein the invariant region is invariant region 4 (IR4) and the means for selectively binding comprises a pair of heavy chains and a pair of light chains, each heavy chain is SEQ ID NO: 7 and each light chain is SEQ ID NO: 8.

15. The composition of matter as recited in claim 14, further comprising a label covalently bound to the peptide, the label selected from a group consisting of a radiolabel and a fluorescent label.

16. A method for labeling a bacterium, the method comprising:
exposing a biological sample to the composition of matter as recited in claim 15, wherein the biological sample comprises the bacterium.

17. An antibody fragment comprising:
a peptide selected from a group consisting of SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8; and
a label covalently bound to the peptide, the label selected from a group consisting of a radiolabel and a fluorescent label.

18. The antibody fragment as recited in claim 17, wherein the antibody fragment has a single peptide selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8 such that the antibody fragment is a single-domain antibody (sdAb).

19. The antibody fragment as recited in claim 17, wherein the antibody fragment has a pair of peptides selected from the group consisting of (1) SEQ ID NO: 1 and SEQ ID NO: 2; (2) SEQ ID NO: 3 and SEQ ID NO: 4; (3) SEQ ID NO: 5 and SEQ ID NO: 6; and (4) SEQ ID NO: 7 and SEQ ID NO: 8.

20. An antibody fragment consisting of:
a peptide selected from a group consisting of SEQ ID NO: 1, SEQ ID NO: 2, SEQ ID NO: 3, SEQ ID NO: 4, SEQ ID NO: 5, SEQ ID NO: 6, SEQ ID NO: 7 and SEQ ID NO: 8; and
a label covalently bound to the peptide, the label selected from a group consisting of a radiolabel and a fluorescent label.


SEQUENCE LISTING (17 sequences)

SEQ ID NO: 1 - protein, 131 aa, synthetic construct
MGWSCIILFLVATATGVHSQSLVESGGGLVQPEGSLTLTCKASGFSFSSGYDMCWVRQAPGKGLEYIACIDAGDDITHYASWVKGRFTVSKTSSTTVTLQLNSLTVADTATYFCGRFWDLWGPGTLVTVSS

SEQ ID NO: 2 - protein, 130 aa, synthetic construct
MGWSCIILFLVATATGVHSSVLTQTPSPVSAAVGGTVTINCQSSQSVYDSTWLGWYQQKPGQPPKLLIYKASNLASGVPSRFKGSGSGTHFTLTISDLECDDAATYYCVGGYSGSVDNWAFGGGTEVVVK

SEQ ID NO: 3 - protein, 128 aa, Synthetic construct
METGLRWLLLVAVLKGVQCQSLEESGGGLFKPTDTLTLTCTVSGIDLSSYAMIWVRQAPGKGLEWIGYIWSSGRIWYASWAKGRFTISRTSTTVDLKLASPTTEDTATYFCARLWDIWGPGTLVTVSS

SEQ ID NO: 4 - protein, 135 aa, Synthetic construct
MDTRAPTQLLGLLLLWLPGATFAQVLTQTPSSVSAAVGGTVTISCQASQSLYNGVNLAWYQQKPGQPPKLLIFGASNLESGVSSRFRGSGSGTQFTLTISGVQCDDAATYYCLGEFSCSSADCLAFGGGTEVVVK

SEQ ID NO: 5 - protein, 127 aa, Synthetic construct
METGLRWLLLVAVLKGVQCQSVEESGGRLVTPGTPLTLTCTVSGFPLSSYSMAWVRQAPGKGLEYIGFINTDGSAYYASWAKGRITISKTSTTVELKITSPTTEDTATYFCGTGNIWGPGTLVTVSS

SEQ ID NO: 6 - protein, 135 aa, synthetic construct
MDTRAPTQLLGLLLLWLPGATFAQVLTQTPSSVSAAVGGTVTINCQASQSVSNNNVLAWFQQKPGQPPKRLIYSALTLDSGVPSRFKGSGSGTHFTLTISGVQCDDAATYYCAGGYDCSSNDCIAFGGGTEVVVK

SEQ ID NO: 7 - protein, 127 aa, Synthetic construct
METGLRWLLLVAVLKGVQCQSVEESGGRLVTPGTPLTLTCTVSGFSLSSYSMGWVRQAPGKGLEYIGMIISNNSTYYASWAKGRITISKTSTTVELKITSPTTEDTATYFCGTGNIWGPGTLVTVSS

SEQ ID NO: 8 - protein, 135 aa, Synthetic construct
MDTRAPTQLLGLLLLWLPGATFAQVLTQTPASVSAAVGGTVTINCQASQSTSNNNALAWFQQKPGQPPKRLIYSALTLDSGVPSRFKGSGSGTHFTLTISGVQCDDAATYYCAGGYDCSSNDCITFGGGTEVVVK

SEQ ID NO: 9 - protein, 22 aa, Synthetic construct
EVSELLDKLVKAVKTAEGASSG

SEQ ID NO: 10 - protein, 21 aa, Synthetic construct
ASVKGIAKGIKEIVEAAGGSE

SEQ ID NO: 11 - protein, 7 aa, Synthetic construct
AGKLFVK

SEQ ID NO: 12 - protein, 22 aa, Synthetic construct
KAAGAVSAVSGEQILSAIVTAA

SEQ ID NO: 13 - protein, 19 aa, Synthetic construct
AEEADNPIAAAIGTTNEDA

SEQ ID NO: 14 - protein, 25 aa, Synthetic construct
MKKDDQIAAAIALRGMAKDGKFAVK

SEQ ID NO: 15 - protein, 13 aa, Synthetic construct
GIAKGIKEIVEAA

SEQ ID NO: 16 - protein, 19 aa, Synthetic construct
KAAGAVSAVSGEQILSAIV

SEQ ID NO: 17 - protein, 8 aa, Synthetic construct
NPIAAAIG